(19) United States
(12) Patent Application Publication
(10) Pub. No.: US 2012/0219536 A1
(43) Pub. Date: Aug. 30, 2012

(54) PURINE-TARGETED DIAGNOSIS AND THERAPY OF WOUNDS

(75) Inventors: Melissa Laura Fernandez, Fitzgibbon (AU); Gary Keith Shooter, East Ipswich (AU)

(73) Assignee: Queensland University of Technology, Brisbane (AU)

(21) Appl. No.: 13/392,340

(22) PCT Filed: Aug. 13, 2010
(86) PCT No.: PCT/AU2010/001037

§ 371 Date: May 7, 2012

(30) Foreign Application Priority Data
Aug. 24, 2009 (AU) 2009904013

(51) Int. Cl. A61K 38/44; A61K 31/7088; C12Q 1/26; G01N 33/573; A61F 13/00; G01N 33/50; A61K 31/519; A61K 31/426; A61K 31/352; A61P 17/02; A61K 38/02; G01N 21/76

(52) U.S. Cl. 424/94.4; 514/18.6; 514/44 R; 435/25; 435/7.4; 436/99; 514/262.1; 436/98; 514/365; 514/456; 602/53

(57) ABSTRACT

Methods of treating a wound, an ulcer and/or a burn responsive to inhibition of xanthine oxidoreductase to thereby reduce uric acid in an animal are provided wherein the methods include topical administration of a therapeutic agent effective for treatment of the wound, the ulcer and/or the burn wherein the therapeutic agent inhibits xanthine oxidoreductase to thereby reduce uric acid in the wound, ulcer and/or burn. Also provided are methods and kits for determining the severity of a wound, a burn and/or an ulcer including the step of detecting one or more of (i) a level of uric acid; (ii) a level of one or more uric acid precursors; and (iii) a presence, or absence of or level of xanthine oxidoreductase.

### FIELD OF THE INVENTION

THIS invention relates to therapy and/or diagnosis of wounds, burns and/or ulcers. More particularly, this invention relates to purine catabolites for use as diagnostic markers and/or therapeutic targets in wounds, burns and/or ulcers.

### BACKGROUND TO THE INVENTION

Wound healing is a dynamic cascade of events that involves the repair, regeneration and remodelling of damaged tissues. A complex, yet sequential process, wound healing is comprised of several critical biochemical events that can be grouped into three overlapping phases: (1) inflammation; (2) proliferation; and (3) remodelling. The inflammatory stage is characterised by the formation of a haemostatic plug, the chemotactic infiltration of leukocytes and the release of cytokines and growth factors that are pivotal for effective wound healing. This is closely followed by the proliferative period which incorporates angiogenesis, synthesis and deposition of extracellular matrix (ECM) and fibroplasia. This leads to the re-epithelialisation stage, commonly referred to as scar formation, followed by the final stage where the ECM is remodeled to restore tensile strength of the regenerated tissue. This interplay of multiple factors demonstrates the necessity for the entire wound healing process to be tightly regulated and why a balanced environment needs to be maintained for effective healing.

Studies have implicated a role for oxygen-derived free radicals in the management of wound healing and in particular venous ulceration (James et al, 2003, Wound Repair and Regeneration, 11: 172). In particular, treatment of wounds with allopurinol has been shown to scavenge oxygen-derived free radicals to thereby stimulate wound healing (Salim, 1991, World Journal of Surgery, 15: 264).

### SUMMARY OF THE INVENTION

Surprisingly the present inventors have found that, at least in part, wound severity is related to xanthine oxidase catalysis of purine precursors to uric acid.

In a broad form, the invention provides a method of diagnosis and/or therapy of a lesion which targets one or more members and/or metabolites of the purine catabolic pathway that lead to uric acid production.

In a first aspect, the invention provides a method of treating a wound, an ulcer and/or a burn responsive to inhibition of xanthine oxidoreductase and uric acid in an animal in need of such treatment, said method including the step of topically administering to said wound, ulcer and/or a burn a therapeutic agent effective for treatment of the wound, the ulcer and/or the burn, wherein said therapeutic agent inhibits xanthine oxidoreductase to thereby reduce uric acid in the wound, the ulcer and/or burn, and thereby treat the wound, ulcer and/or the burn in said animal.

In preferred embodiments, the method of the first aspect further includes the step of determining whether the animal has an elevated level of xanthine oxidoreductase and/or uric acid in the wound, the ulcer and/or the burn.

Preferably, the method of the first aspect further includes the step of promoting clearance of uric acid from the wound, the ulcer and/or the burn. More preferably, promoting clearance of uric acid is by way of a chemical stimulus or physical stimulus.

Preferably, the chemical stimulus is a biochemical stimulus. More preferably, the biochemical stimulus is by way of enzymatic catalysis.

More preferably, the method further includes the step of applying a physical stimulus to the wound, the ulcer and/or the burn to promote clearance of uric acid.

Even more preferably, the physical stimulus is compression. Yet even more preferably, the physical stimulus is applied by a compression bandage.

In a second aspect, the invention provides a method of determining the severity of a wound, a burn and/or an ulcer in an animal, said method including the step of detecting one or more of the following:
(i) a level of uric acid, wherein an increased level correlates with increased wound severity;
(ii) a level of one or more uric acid precursors, wherein a decreased level correlates with increased wound severity; and
(iii) a presence or absence of, or level of, xanthine oxidoreductase, wherein an increased level correlates with increased wound severity
in a cell, a tissue and/or a fluid of the wound, the burn and/or the ulcer to thereby determine the severity of the wound, the burn and/or the ulcer.

In a third aspect, the invention provides a kit for determining the severity of a wound, a burn and/or an ulcer, said kit comprising one or more reagents for detecting one or more of the following:
(i) a level of uric acid, wherein an increased level correlates with increased wound severity;
(ii) a level of one or more uric acid precursors, wherein a decreased level correlates with increased wound severity; and
(iii) a presence or absence of, or level of, xanthine oxidoreductase, wherein an increased level correlates with increased wound severity,
in a cell, a tissue and/or a fluid of the wound, the burn and/or the ulcer to thereby determine the severity of the wound, the burn and/or the ulcer.

In preferred embodiments of the second and third aspect, items (i) and (ii) are detected.

Preferably, the second and third aspects correlates a ratio of (i) relative to (ii) with increased wound severity. Even more preferably, the ratio is an elevated ratio.

In a fourth aspect, the invention provides a pharmaceutical composition for use in treating a wound, a burn and/or an ulcer responsive to inhibition of xanthine oxidoreductase and uric acid in an animal in need of such treatment, wherein said pharmaceutical composition comprises a therapeutic agent effective for treatment of the wound, the ulcer and/or the burn, wherein said therapeutic agent inhibits xanthine oxidoreductase to thereby reduce uric acid in the wound, the ulcer and/or burn, together with a pharmaceutically acceptable diluent, carrier or excipient.

In one form, the invention provides a pharmaceutical composition when used for treating a wound, an ulcer and/or a burn responsive to inhibition of xanthine oxidoreductase and uric acid in an animal in need of such treatment.

In another form, the invention provides a pharmaceutical composition when used in a method of treating a wound, an ulcer and/or a burn responsive to inhibition of xanthine oxidoreductase and uric acid according to the first aspect.

In yet another form, the invention provides use of a pharmaceutical composition in a method of treating a wound, an ulcer and/or a burn responsive to inhibition of xanthine oxidoreductase and uric acid according to the first aspect.

In preferred embodiments of any one of the aforementioned aspects, xanthine oxidoreductase is the xanthine oxidase form.

In other preferred embodiments of any one of the aforementioned aspects, xanthine oxidoreductase is the xanthine dehydrogenase form.

According to preferred embodiments of any one of the aforementioned aspects, the therapeutic agent either directly or indirectly inhibits xanthine oxidoreductase. In particularly preferred embodiments, the inhibitor is a selective inhibitor.

Preferably, the therapeutic agent is selected from the group consisting of an isolated protein, an isolated peptide, an isolated nucleic acid and a small organic molecule. The isolated nucleic acid may encode an inhibitor which is a protein or alternatively, the isolated nucleic acid may itself have inhibitory activity such as, but not limited to, an RNAi molecule or a ribozyme.

In preferred embodiments, the therapeutic agent which is a small organic molecule is an analogue and more preferably, the analogue is a purine analogue.

Even more preferably, the purine analogue is selected from the group consisting of allopurinol, oxypurinol, alloxanthine, adenosine and thiopurinol.

In other preferred embodiments, an inhibitor of xanthine oxidase and/or uric acid is an antagonist.

Accordingly, an inhibitor of xanthine oxidase is a flavonoid. Suitable flavonoids include febuxostat, myricetin, kaempferol and quercetin, but without limitation thereto. In particularly preferred embodiments, the flavonoid is febuxostat.

Preferably, the animal is a mammal.

More preferably, the mammal is a human.

It will also be appreciated that treatment methods, diagnostic methods and pharmaceutical compositions may be applicable to prophylactic or therapeutic treatment of mammals, inclusive of humans and non-human mammals such as livestock (e.g. horses, cattle and sheep), companion animals (e.g. dogs and cats), laboratory animals (e.g. mice rats and guinea pigs) and performance animals (e.g. racehorses, greyhounds and camels), although without limitation thereto.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF FIGURES

In order that the invention may be readily understood and put into practical effect, preferred embodiments will now be described by way of example with reference to the accompanying:

FIG. 1 Separation of chronic wound fluid (CWF) by Size Exclusion Chromatography. Parts (a) and (b) show UV chromatograms of gel filtration standards (Bio-Rad) and overlayed traces of serum and CWF samples, respectively. Peaks representing low molecular weight compounds that eluted towards the end of the chromatography run are highlighted in (b). These peaks elicited a strong absorbance in the CWF profile in contrast to the human serum profile. Bio-Rad gel filtration standards ranging from 1.35 to 670 kDa provide an estimate of molecular weights across serum and CWF chromatograms.

FIG. 2 Analysis of fractionated CWF by SDS PAGE electrophoresis. The chromatography profile of wound fluid highlights the fractions that were selected for further analysis by SDS PAGE. The silver stained gel demonstrates the separation of protein in these selected fractions. Of note, no protein was visualised in fractions 7, 8 and 9 although their absorbance intensity in the chromatography profile was similar to fraction 3, 4 and 6. Pre-stained molecular weight marker with bands between 10 and 250 kDa in size (Precision Dual Colour Marker, Bio-Rad) was used to estimate protein sizes.

FIG. 3 Analysis of Wound Fluid at 254 nm and 280 nm. De-proteinised CWF was separated using reverse phase chromatography. The chromatograms generated were monitored at both 254 nm (a) and 280 nm (b). The peak highlighted in the two profiles elicited a stronger response at 280 nm compared to 254 nm.

FIG. 4 HPLC chromatograms of purine metabolite in a calibration solution. Chromatograms of a mixture of hypoxanthine, uric acid, xanthine, adenosine and inosine monitored at 254 nm (a) and 280 nm (b). The calibration solution contained 100 μmol/L of each purine.

FIG. 5 Chromatograms demonstrating the amount of purine standards to be incorporated into CWF. The concentrations of purine standards to be added to wound fluid were determined to be 20 μmol/L of hypoxanthine, 5 μmol/L of uric acid and inosine and 2.5 μmol/L of xanthine and adenosine. (a) depicts a representative chromatogram of a mixture of these purine standards and (b) depicts overlayed traces of the standards in relation to CWF.

FIG. 6 Evidence of purine metabolites in CWF. Overlayed traces comparing the added purine standard profile with chromatograms of spiked wound fluid (a) and unprocessed CWF (b). Evaluation of these chromatograms together provides evidence that suggests the presence of purines in CWF.

FIG. 7 Evidence of active xanthine oxidase (XO) in CWF. Overlayed traces demonstrating activity of XO in pooled wound fluid (a) by the production of uric acid and (b) by the production of the metabolite oxypurinol during inhibition with allopurinol. CWF supplemented with either xanthine or allopurinol (10 μmol/L) was incubated for up to 2.5 hr and the production of uric acid and oxypurinol determined by A₂₈₀ and A₂₅₄ respectively. This activity of XO in CWF was confirmed by detection of uric acid and oxypurinol production.

FIG. 8 Evidence of Xanthine Oxidase in clinically worse ulcers. Elevated levels of xanthine oxidase was detected in chronic wound fluid from clinically worse ulcers (n=10, PUSH 10-16) compared to human serum. Levels were expressed as μM of uric acid/mg of Total Protein.

FIG. 9 Uric Acid and Purine Precursors in CWF (a) Elevated Uric Acid Correlates with Wound Chronicity. Levels were expressed as amount of uric acid as a percentage of total purines±SEM. The amount of uric acid (UA) was significantly increased (p<0.0001) in the clinically worse ulcers (10-14) compared to the lower PUSH score group (4-9). Statistical significance was determined by Mann Whitney non-parametric test. (b) Reduced Purine Precursors of Uric Acid Correlates with Wound Chronicity. Levels were expressed as concentration of the precursor purines (sum totals of adenosine, inosine, xanthine and hypoxanthine) (μM/mg of Total Protein)±SEM. The amount of precursor purines was significantly higher (p<0.0001) in the lower PUSH score group (4-9) compared to the clinically worse ulcers (10-14). Statistical significance was determined by the Mann-Whitney non-parametric test.

FIG. 10 Correlation plot of Chronic Wound Fluid Samples (n=55).

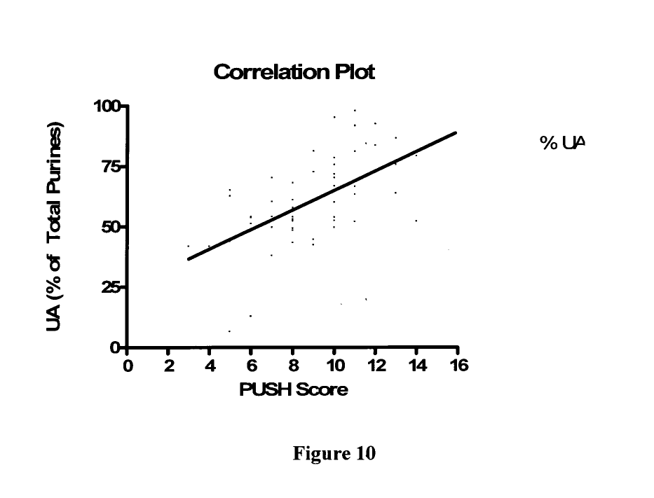

### DETAILED DESCRIPTION OF THE INVENTION

The present invention is predicated, at least in part, on the finding that purine metabolites and in particular an elevated ratio of uric acid to total purines is correlative with wound severity. Moreover, it is also postulated by the present inventors that xanthine oxidase activity is also a factor in wound severity due in part, to the finding that xanthine oxidase activity is inhibited by allopurinol in chronic wound fluid.

Therefore it is proposed that uric acid may be an important indicator for management of lesions in the form of wounds; burns and/or ulcers and may be useful as a therapeutic target for the treatment of these lesions and/or for diagnosis of these lesions.

It will be understood that the term “wound” is a trauma situated or derived from any cell, tissue or organ of the body and is inclusive of venous wounds, arterial wounds, organ wounds and topical skin wounds. “Wounds” also include a wound resulting from or associated with a particular disease, disorder or condition. In a particularly preferred embodiment, the wound is a diabetic wound.

The term “ulcer” includes within its scope any ulceric lesion inclusive of pressure ulcers.

In broad aspects, the invention provides methods of therapy which utilise a therapeutic agent effective for treatment of the wound, the ulcer and/or the burn, wherein the therapeutic agent inhibits xanthine oxidoreductase to thereby reduce uric acid levels.

It will be understood by a skilled addressee that xanthine oxidoreductase (XOR) refers to broader classification of the enzyme which catalyses the oxidation of hypoxanthine to xanthine and the oxidation of xanthine to uric acid. XOR can exist in two interconvertible forms, xanthine oxidase (XO) and xanthine dehydrogenase (XDH). Both forms convert xanthine to uric acid. During this process, XO uses O₂ and H₂O as co-substrates and liberates H₂O₂ as a biproduct. For XDH, the co-substrates are NAD+ and H₂O with the biproducts being NADH and H+. Preferred embodiments of the present invention relate to either XO or XDH. Particularly preferred embodiments relate to xanthine oxidase.

In the context of the present invention, by “inhibit”, “inhibition”, “inhibited”, “inhibitory” or “inhibitor” is meant a therapeutic agent which at least partly interferes with, inhibits, reduces, blocks or hinders one or more biological activities or effects of xanthine oxidoreductase and preferably xanthine oxidase, either by a direct or an indirect mechanism. It will also be appreciated that in certain preferred embodiments an inhibitor used in the present invention is a selective inhibitor of xanthine oxidoreductase and more preferably, xanthine oxidase. “Inhibitors” of the present invention includes within its scope the terms “agonist”, “analogue” and “antagonist”.

Accordingly, suitable therapeutic agents and/or inhibitors of the invention may be peptides, proteins inclusive of antibodies, nucleic acids, protein complexes or other organic molecules, a small organic molecule, with a desired biological activity and half-life.

Proteins and peptides may be useful in native, chemical synthetic or recombinant synthetic form and may be produced by any means known in the art, including but not limited to, chemical synthesis, recombinant DNA technology and proteolytic cleavage to produce peptide fragments.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

Useful therapeutic agents of the present invention includes agents typically used for treatment of diseases or disorders related to elevated levels of uric acid such as gout and other hyperuricemic disorders.

Therefore it is contemplated that particular preferred embodiments utilise purine analogues or derivatives that inhibit xanthine oxidoreductase and more preferably xanthine oxidase and is inclusive of hypoxanthine and xanthine-based analogues, although without limitation thereto. In particularly preferred embodiments, the purine analogue is adenosine.

In certain preferred embodiments, the purine analogue is a structural isomer of hypoxanthine and in turn, inhibits xanthine oxidase. Allopurinol (can be otherwise known as 1,4-dihydropyrazolo[4,3-d]pyrimidin-7-one as well as other suitable synonyms known to a person of skill in the art) is a particularly preferred purine analogue.

The invention also contemplates use of xanthine analogues, and more preferably, alloxanthine (can be otherwise known as 1,2-dihydropyrazolo[4,3-e]pyrimidine-4,6-dione as well as other suitable synonyms known to a person of skill in the art) as an inhibitor.

Other types of inhibitors include imidazole and triazole derivatives, and flavonoids, although without limitation thereto.

Suitable flavonoid inhibitors include febuxostat (can otherwise be known as 2-[3-cyano-4-(2-methylpropoxy)phenyl]-4-methyl-1,3-thiazole-5-carboxylic acid as well as other suitable synonyms known to a person of skill in the art), myricetin, kaempferol and quercetin, although without limitation thereto. Reference is made to Iio et al 1985 Agric Biol Chem 49 2173-76, which provides non-limiting examples of suitable flavonoids. Febuxostat is a particularly preferred flavonoid.

In certain preferred embodiments, the inhibitor has an IC₅₀ of less than about 1 mM, more preferably less than about 0.5 mM, about 0.2 mM and about 0.1 mM. In other certain preferred embodiments that relate to xanthine oxidase inhibitors, the xanthine oxidase inhibitor has an IC₅₀ of less than about 1 mM, more preferably less than about 0.5 mM, about 0.2 mM and about 0.1 mM.

In other particularly preferred embodiments, the inhibitor is not pentoxifylline. In certain preferred embodiments, the xanthine oxidase inhibitor is not pentoxifylline.

It will be further appreciated that for convenience, the trivial rather than the correct systematic nomenclature of compounds may be used throughout this specification.

### Therapeutic Uses

In particular aspects, the present invention provides methods of treating burns, wounds and/or ulcers which are responsive to inhibition of xanthine oxidoreductase and uric acid in an animal in need of such treatment, by administering therapeutic agents as hereinbefore described. In preferred embodiments, methods of treatment relate to topical administration. It will be appreciated that topical administration primarily causes a localised effect. In preferred forms that relate to topical administration, administration is by way of surface administration and more preferably by direct application to a skin surface of a burn, wound and/or ulcer.

The methods of the present invention are particularly aimed at treatment of mammals, and more particularly, humans. However, it will also be appreciated that the invention may have veterinary applications for treating domestic animals, livestock, laboratory animals and performance animals as would be well understood by the skilled person.

In particular embodiments, therapeutic treatments may utilize a therapeutic agent in association with, or as a component of, a biomaterial, biopolymer, inorganic material such as fluorohydroxyapatite, surgical implant, prosthesis, wound, ulcer or burn dressing, compress, bandage or the like suitably impregnated, coated or otherwise comprising the therapeutic agent.

In preferred embodiments, the methods of treatment of the present invention are localised at the site of the wound, ulcer and/or burn. Localised treatment includes, but is not limited to, treatment with bandages, compress, dressings and the like.

The therapeutic methods of the invention may further include the step of promoting clearance of uric acid from the site of the wound, burn and/or ulcer. In preferred embodiments, a physical or mechanical means is employed to increase or promote clearance of uric acid. In particularly preferred embodiments, the mechanical means is by way of application of compression or a compressive pressure. Other techniques may incorporate the use of debridment and/or inclusion of antioxidants or reducing agents containing free sulfhydryl groups.

In other preferred embodiments that relate to promoting clearance of uric acid, a biochemical intervention that specifically promotes clearance of uric acid may be used. In certain embodiments, an enzyme which catalyses the breakdown of uric acid may be employed. Non-limiting examples of suitable enzymes include oxidases In certain preferred embodiments, the enzyme is uricase.

It will be appreciated that the therapeutic agents of the present invention may form part of a pharmaceutical composition or may be administered as part of a pharmaceutical composition. Modes of administration may be by way of microneedle injection into specific tissue sites, such as described in U.S. Pat. No. 6,090,790, topical creams, lotions or sealant dressings applied to wounds, burns or ulcers, such as described in U.S. Pat. No. 6,054,122 or implants which release the composition such as described in International Publication WO99/47070.

Suitably, pharmaceutical compositions further comprise a pharmaceutically acceptable carrier, diluent or excipient.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991) which is incorporated herein by reference.

Any safe route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight, severity of conditions being treated, the agents being employed and general health condition thereof, factors that will depend on the judgement of the practitioner. The use of either daily administration or post-periodic dosing may be employed. Thus, generally, and without limitation, the pharmaceutical compositions herein may contain, per unit dosage unit, (e.g., tablet, capsule, powder, injection, suppository, teaspoonful and the like) of from about 0.01-3000 mg of an active agent or any range therein.

### Diagnosis

Broadly, the present invention is directed towards diagnostic methods to determine the severity of a wound, an ulcer and/or burn, which utilise uric acid, xanthine oxidoreductase and uric acid biosynthetic precursors, or combinations thereof.

In certain preferred embodiments, methods of diagnosis of the present invention may be used as a prognostic marker of healing, or potential healing of a wound, an ulcer and/or a burn. In other preferred embodiments, the diagnostic methods of the present invention may be utilised as a marker of potential responsiveness to treatment with a therapeutic agent and in particular a therapeutic agent that inhibits xanthine oxidoreductase.

It will be appreciated that in by “detect”, “detection” or “detecting”, is meant to assay, measure or otherwise identify a marker of interest.

The diagnostic methods of the present invention include measurement of a relative level or an absolute level.

In a particular embodiment, the invention contemplates detecting a level of uric acid and/or a level of one or more uric acid precursors. According to these embodiments, the level may be an absolute level or alternatively, a relative level particularly in the form of a ratio.

In preferred embodiments, the level of one or more uric acid precursors in a sample is an absolute level.

Preferably, the diagnostic method of the invention detects an absolute level of uric acid in a sample to indicate the severity of a wound, a burn and/or an ulcer.

In embodiments which encompass detection of a relative level of uric acid, the diagnostic method includes measurement of a ratio of uric acid relative to one or more uric acid precursors in a sample, which indicates the level of severity of a wound, a burn and/or an ulcer. Preferably, the ratio is an elevated ratio of uric acid relative to one or more uric acid precursors. An elevated ratio of uric acid to one or more uric acid precursors can be correlated to or associated with an increase in severity of a wound, a burn and/or an ulcer.

In a preferred embodiment that relate to diagnostic methods the invention relates to a method of determining the severity of a wound, a burn and/or an ulcer in an animal, said method including the step of detecting one or more of the following:
(i) a level of uric acid, wherein a relatively increased level correlates with increased wound severity;
(ii) a level of one or more uric acid precursors, wherein a relatively decreased level correlates with increased wound severity; and
(iii) a presence or absence of, or level of, xanthine oxidoreductase, wherein a relatively increased level correlates with increased wound severity
in a cell, a tissue and/or a fluid of the wound, the burn and/or the ulcer to thereby determine the severity of the wound, the burn and/or the ulcer.

In other preferred embodiments, a level of uric acid may be expressed as a percentage of total purine precursors.

Uric acid precursors are inclusive of precursor purines such as adenosine, inosine, guanine, xanthine and hypoxanthine, and other precursors of the uric acid biosynthetic pathway.

In other preferred embodiments that relate to detecting uric acid levels, a biological assay and in particular, an enzymatic assay may be particularly suitable. In certain embodiments, an enzyme which catalyses the breakdown of uric acid may be employed. Non-limiting examples of suitable enzymes include oxidases. In certain preferred embodiments, the enzyme is uricase. It will be appreciated that diagnostic methods of the present invention which include measuring uric acid may employ ready available commercial kits such as the Uric Acid Assay Kit from Biovision.

It will be appreciated that small organic molecules such as purines and uric acid may be detected by a variety of techniques as are well known in the art, inclusive of biophysical techniques such as mass spectrometry, chromatographic methods including high-performance liquid chromatography and reverse-phase liquid chromatography, light emission such as chemiluminescence and UV absorption, although without limitation thereto.

Accordingly, diagnostic methods of the present invention may be protein-based. Protein-based techniques applicable to the invention are well known in the art and include western blotting, ELISA, two dimensional protein profiling, protein arrays, immunoprecipitation, an enzymatic assay, mass spectrometry, immunohistochemistry, radioimmunoassays and colorimetric detection, although without limitation thereto.

In a particularly preferred embodiment, a level of one or more uric acid precursors in a sample may be detected by contacting said sample with a xanthine oxidase which, in turn, results in the formation of uric acid and hydrogen peroxide. The hydrogen peroxide may be measured using standard colourimetric detection techniques, or any other applicable technique as is known in the art.

According to these embodiments, the xanthine oxidase is bound directly to a substrate or in alternative embodiments, is bound to an antibody for example as part of an ELISA format, although without limitation thereto. In particularly preferred embodiments, the diagnostic methods of the invention may take the form of an enzyme-linked test strip in which the xanthine oxidase is coupled to a solid-support. By way of example, a fluid from a wound, a burn and/or an ulcer can be applied to the test strip and if one or more uric acid precursors are present in the sample, xanthine oxidase will convert the precursors to uric acid and hydrogen peroxide. The hydrogen peroxide may then be detected by horseradish peroxidase colourimetric reaction, or the like. This arrangement is similar to the test strips for determination of glucose in urine or blood in which glucose oxidase is immobilized on the test strip.

It will also be appreciated that diagnostic methods of the invention may be used alone or combined with other forms of clinical diagnosis to improve the accuracy of diagnosis. By way of example only, techniques such as The Pressure Ulcer Scale Of Healing (PUSH tool) and use of other clinically relevant data related to healing progress such as oedema, eczema and signs of infection may also be employed to track healing of wounds, burns and/or ulcers.

The present invention also encompasses a kit for colourimetric detection which may, for example, detect generation of hydrogen peroxide using horseradish peroxidase. As is well known in the art, horseradish peroxidase catalyzes the conversion of chromogenic substrates (e.g. TMB, DAB) into coloured molecules. It is also contemplated that a kit may use other detection systems using chemiluminescent substrates (e.g. SuperSignal, ECL) for the production of light. Fluorometric detection systems may also be employed, particularly for the detection of hydrogen peroxide wherein hydrogen peroxide may oxidise a non-fluorescent substrate such as 10-Acetyl-3,7-dihydroxyphenoxazine (ADHP) or Amplex® Red to produce a fluorescent product that can be monitored by spectrometry. The aforementioned are non-limiting examples.

It is envisaged that in embodiments that relate to detection of xanthine oxidoreductase, expression may be analysed by either protein-based or nucleic acid-based techniques.

Alternatively, relative protein expression levels may be determined by other protein-based methods which include immunoassays, for example ELISA and immunoblotting to detect relative expression levels of one or more of said proteins.

The invention further contemplates use of microarray technology to determine the expression pattern profile of a wound, burn and/or ulcer in order to analyse the presence, absence of, or level or xanthine oxidoreductase in a sample.

Proteomic pattern analysis provides an alternative diagnostic method which is particularly useful for global expression pattern analysis of proteins. In particular embodiments, a plurality of said proteins may be used in a protein library displayed in a number of ways, e.g., in phage display or cell display systems or by two-dimensional gel electrophoresis, or more specifically, differential two-dimensional gel electrophoresis (2D-DIGE). These particular embodiments may generally be referred to as “proteomic” or “protein profiling” methods, such as described in Chapters 3.9.1 and 22 of CURRENT PROTOCOLS IN PROTEIN SCIENCE Eds. Coligan et al., John Wiley & Sons NY USA (1996-2002).

In embodiments relating to protein arrays, preferably a xanthine oxidoreductase protein of the invention is located at an identifiable address on the array. Preferably, the protein array comprises a substrate to which is immobilized, impregnated, bound or otherwise coupled breast cancer-associated protein, or a fragment thereof. The substrate may be a chemically-derivatized aluminum chip, a synthetic membrane such as PVDF or nitrocellulose, a glass slide or microtiter plates.

Detection of substrate-bound proteins may be performed using mass spectrometry, ELISA, immunohistochemistry, fluorescence microscopy or by colorimetric detection.

A person of skill in the art will the diagnostic methods of the invention may involve measuring expression levels of a nucleic acid encoding a xanthine oxidoreductase and more preferably, xanthine oxidase. In this regard, nucleotide sequence variations in a promoter, for example, may affect the steady state levels of a xanthine oxidoreductase gene transcript in one or more cells of an affected or predisposed individual.

It is also contemplated that relative levels of nucleic acids may be measured and/or compared in the diagnostic methods of the present invention. Measurement of relative levels of a nucleic acid level compared to an expressed level of a reference nucleic acid may be conveniently performed using a nucleic acid array. Nucleic acid array technology has become well known in the art and examples of methods applicable to array technology are provided in Chapter 22 of CURRENT PROTOCOLS IN MOLECULAR BIOLOGY Eds. Ausubel et al. (John Wiley & Sons NY USA 1995-2001).

An array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

Reference is also made to Affymetrix nucleic acid array systems such as described in U.S. Pat. No. 5,858,659 and U.S. Pat. No. 6,300,063 which provide specific teaching in relation to nucleic acid array-based detection of disease-related polymorphisms. In another particular form of this embodiment, quantitative or semi-quantitative PCR using primers corresponding to xanthine oxidoreductase-encoding nucleic acids may be used to quantify relative expression levels of a xanthine oxidoreductase nucleic acid to thereby determining the severity of a wound, ulcer and/or burn.

PCR amplification is not linear and hence end point analysis does not always allow for the accurate determination of nucleic acid expression levels. Real-time PCR analysis provides a high throughput means of measuring gene expression levels. It uses specific primers, and fluorescence detection to measure the amount of product after each cycle. Hydridization probes utilise either quencher dyes or fluorescence directly to generate a signal. This method may be used to validate and quantify nucleic acid expression differences in cells or tissues obtained from sufferers compared to cells or tissues obtained from non-sufferers.

It will be appreciated that in certain preferred embodiments of diagnostic methods of the present invention, a level, preferably an increased level and more preferably a relatively increased level, may be a single-fold difference, a two-fold difference, a three-fold difference, a four-fold difference, a five-fold difference, a six-fold difference, a seven-fold difference, an eight-fold difference, a nine-fold difference and equal to or greater than a ten-fold difference.

In preferred embodiments that relates to kits, the present invention provides a kit for determining the severity of a wound, a burn and/or an ulcer in an animal, said method including the step of detecting one or more of the following:

(i) a level of uric acid, wherein a relatively increased level correlates with increased wound severity;

(ii) a level of one or more uric acid precursors, wherein a relatively decreased level correlates with increased wound severity; and

(iii) a presence or absence of, or level of, xanthine oxidoreductase, wherein a relatively increased level correlates with increased wound severity

in a cell, a tissue and/or a fluid of the wound, the burn and/or the ulcer to thereby determine the severity of the wound, the burn and/or the ulcer.

So that the invention may be readily understood and put into practical effect, the following non-limiting Examples are provided.

### EXAMPLES

### Example 1

### Evidence of Purine Metabolites in CWF

### Analysis of CWF by Size Exclusion Chromatography

Gel filtration chromatography was used to separate protein and peptides in CWF and serum (Sigma-Aldrich) on the basis of size in an attempt to capture low molecular weight compounds. Briefly, samples were analysed on the BioLogic Duoflo (Bio-Rad), a fully automated dual pump fast protein liquid chromatography system (FPLC), which was linked to a BioFrac fraction collector (Bio-Rad). The Tricorn™ Superose™ 6 high performance column [13±2 μm, 10 mm×300 mm] with a molecular mass fractionation in the range of 5-500 kDa was used to separate proteins and peptides present in CWF and serum. The eluent selected was 150 mM Ammonium Bicarbonate pH 7.8 which was compatible with downstream analysis. CWF was diluted one in two with buffer to a total volume of 400 μl, then centrifuged to remove any particulate matter to avoid fouling of the column. Compounds were eluted isocratically from the column over a period of 56 min as the flow rate was maintained at 0.5 mL/min. Elution profiles were monitored at A₂₈₀ and fractions were collected at 1 min intervals. Fractions were stored at −20° C. until further analysis.

### SDS PAGE

Several fractions from across the CWF profile were selected for further separation using SDS PAGE with the purpose of visualising protein content. Fractions were separated on pre-cast NuPAGE® Novex® Bis-Tris 4-12% minigels (Invitrogen). As per manufacturer's instructions, samples were added to NuPAGE® loading buffer (Invitrogen) containing NuPAGE® reducing agent (Invitrogen) and incubated at 70° C. for 10 min prior to loading. The gradient gel was electrophoresed in 1×NuPAGE® MES SDS running buffer (Invitrogen) at 200 V for 35 min. A pre-stained molecular weight marker between 10 and 250 kDa in size (Precision Dual Colour Marker, Bio-Rad) was used to identify appropriate electrophoresis time and to estimate protein sizes. Gels were stained with Pierce silver staining kit to visualise protein bands.

### Detection of Purine Metabolites in CWF

Reverse phase (RP) HPLC/UV analysis of purine metabolites in CWF was performed using a method based on that described by Kojima et al (1986). Stock standard solutions of adenosine, inosine, hypoxanthine, uric acid and xanthine-were prepared at 6 mmol/L in 20 mmol/L sodium hydroxide (NaOH) solution. These standards were stored at −20° C., and were thawed on ice prior to analysis. Equal volumes of all five standards were added and diluted with deionised distilled water to a final concentration of 100 tμmol/L. A sub-aliquot of pooled CWF sample was thawed and de-proteinised using Microcon® 10 kDa cut-off centrifugal filters (Millipore) according to manufacturer's instructions. The resulting filtrates were then injected on the HPLC column.

Standards and the CWF sample were analysed using the BioLogic Duoflo chromatography system (Bio-Rad) with a UV detector (254 nm/280 nm) and a Polaris® C18 analytical column 5 μm, 250×4.6 mm i.d. (Varian). The mobile phase was 40 mmol/L KH₂PO₄ (potassium phosphate) buffer, pH 2.2 containing 2% methanol. The compounds were eluted using an isocratic flow rate maintained at 0.5 mL/min. Elution profiles were monitored at either A₂₅₄ or A₂₈₀. The column was regenerated in 80% acetonitrile/40 mmol/L potassium phosphate, pH 2.2, following each run to ensure that there was no carry-over between runs.

### Results

### Evidence of Non-Proteinaceous Agents in CWF

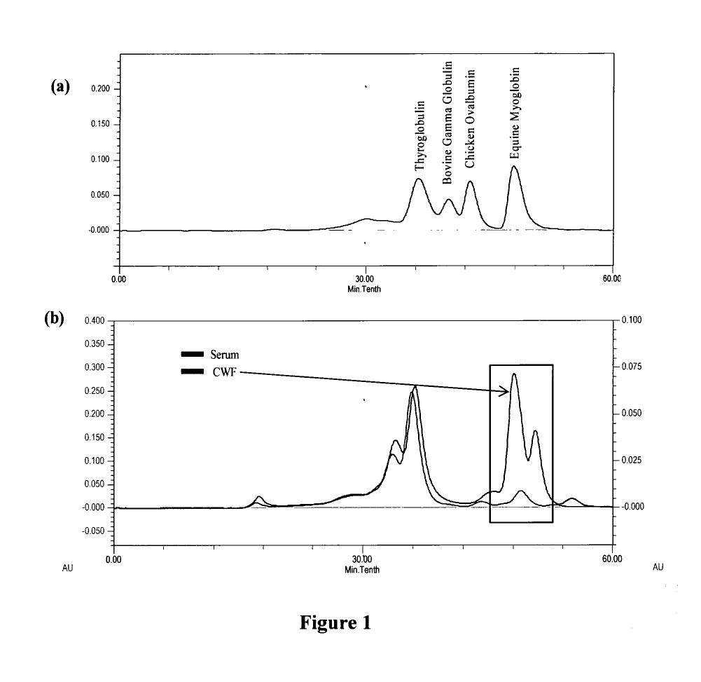

Size exclusion chromatography was used to separate large proteins in CWF from the smaller molecules, with the intent to isolate these smaller molecules (>30 kDa) for further analysis. Proteins in wound fluid were separated on the basis of size as they pass through the column, with large molecular weight molecules moving quickly through the column bed and eluting first, followed by the small molecular weight molecules. The separation profiles of serum and CWF samples were monitored at A₂₈₀ as shown in FIG. 1.

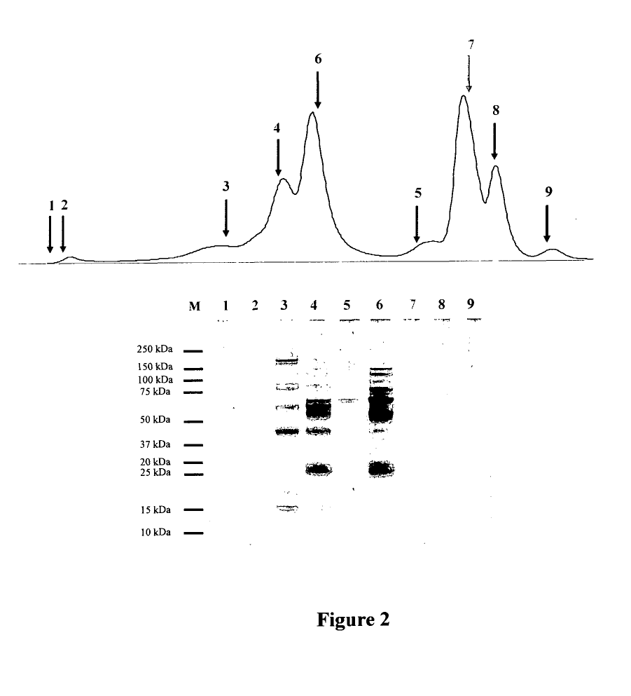

Bio-Rad gel filtration standards were analysed and generated profiles that demonstrated good separation and elution peaks as shown in FIG. 1a. In addition, serum was also examined as a control owing to its close association with CWF with regard to protein composition (FIG. 1b). A pool of CWF was then analysed under the same conditions and displayed a similar high molecular weight profile to serum (FIG. 2b). However, based on the absorbance readings, it was observed that the protein composition was much higher in serum when compared to CWF. More interestingly, the separation of these biological fluids revealed the presence of a couple of peaks that eluted towards the latter stages of the chromatography run, characteristic of low molecular weight compounds, which are highlighted in FIG. 1b. It was noted, specifically in the CWF profile, that these peaks elicited a strong absorbance when compared to those that are present in serum, especially considering the higher protein concentration of serum.

In order to visualise the separation of proteins/peptides in CWF, as well as investigate the peaks identified in the box in FIG. 1b, several fractions from across the CWF profile were selected for analysis using SDS PAGE. The CWF fractions that were selected for gel electrophoresis are highlighted in FIG. 2. Equal volumes of each fraction were electrophoresed on a gradient gel under reducing conditions. The silver stained gel revealed the presence of protein in lanes 3-6 corresponding to the peaks eluted during gel filtration chromatography. Of interest, low molecular weight compounds were evident in fractions 3 and 4, yet these fractions were expected to mainly be comprised of higher molecular weight proteins. Nevertheless, exposure to the reducing/denaturing conditions in the SDS-PAGE analysis will disrupt intra- and inter-molecular bonds/associations, which will contribute to the appearance of low molecular weight protein subunits in these fractions. In addition, it was expected that the bulk of the smaller compounds would be eluted in fraction 5, yet, there was little evidence of protein in this fraction (FIG. 2). One plausible explanation for these unanticipated results is that the constituents of wound fluid may interact with each other, potentially resulting in protein aggregation; for instance, albumin is known to sequester proteins. Surprisingly, however, the SDS PAGE gel provided no evidence of proteinaceous material in peaks 7, 8 and 9. This outcome was unexpected since the intensity of this peak (7) was similar to that of peak (6) which contained a complex mixture of high abundant proteins. This led us to believe that the low molecular weight peaks that strongly absorb at 280 nm may represent purine metabolites such as uric acid.

### Evidence of Strongly Absorbing Material at 254 nm

Following confirmation that the interfering substances were not DNA, pooled wound fluid was examined using the NanoDrop ND-1000 (NanoDrop, Wilmington, Del., USA). Unprocessed CWF, human serum (Sigma-Aldrich) and acute wound fluid (AWF) samples were analysed on the NanoDrop within a wavelength range of 220-800 nm to assess if wound fluid interacted at any other wavelength. In contrast to serum and AWF, continuous wavelength monitoring of wound fluid revealed that material present in wound fluid strongly absorbed at approximately 260 nm and 280 nm (Data not shown).

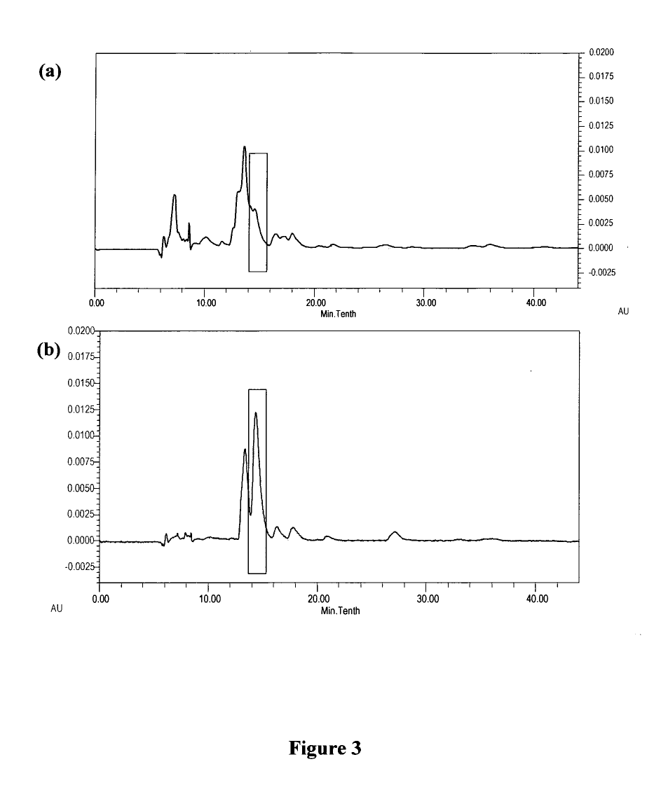

Continuing evidence that these fractions contained material that strongly absorbed at 280 nm led us to believe that the low molecular weight peaks that strongly absorb at 280 nm may perhaps be uric acid. Thus, the wound fluid was analysed using reverse phase chromatography as described herein. Based on our previous findings, which indicated that these substances were not protein, CWF was de-proteinised using 10 kDa filters in order to avoid any interference. The resulting filtrates were separated isocratically and the profiles were monitored at both 254 nm and 280 nm. The results obtained demonstrate the presence of material in CWF that absorbs at both 254 nm and 280 nm (FIG. 3). However, whilst the same sample was analysed at two different wavelengths, two completely different traces emerged. More specifically, on closer inspection of these traces it was evident that there was one striking difference between the two profiles. As highlighted in FIG. 3, a peak at similar elution time strongly absorbed at 280 nm (b) and absorbed with less intensity at 254 nm (a). Therefore, from both the initial observations using the NanoDrop, followed by analysis of CWF using reverse phase chromatography, it appeared that these substances interacted strongly at 280 nm rather than 254 nm. It was therefore hypothesized that these substances could be part of the purine catabolic pathway as these naturally occurring aromatic compounds are known to absorb at both 254 nm as well as 280 nm. However, there were slight shift in retention times between the two profiles making it difficult to draw any conclusions between the two profiles.

### Evidence of Purine Metabolites in CWF

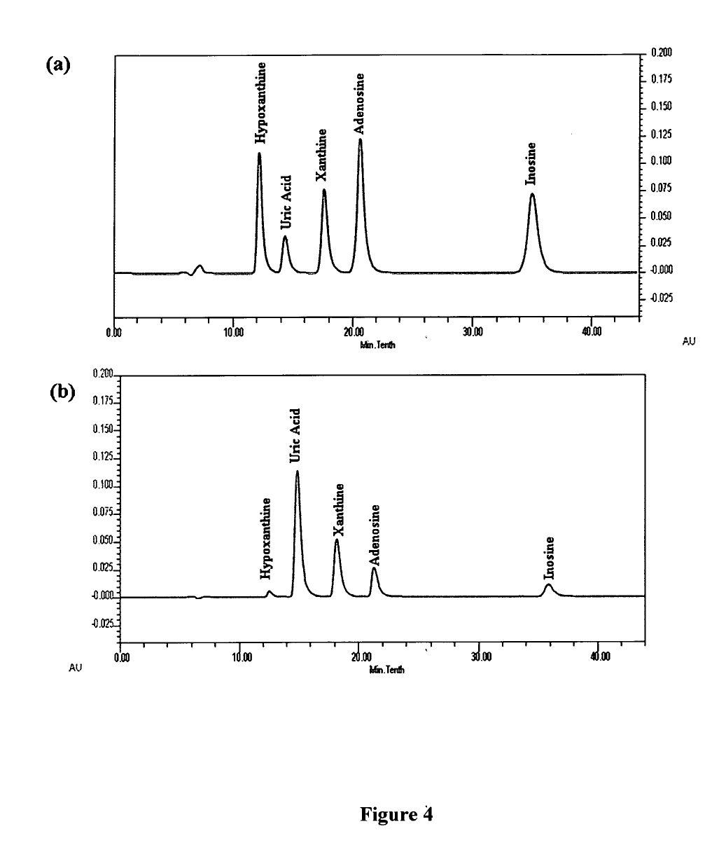

A HPLC/UV method previously described was used to verify whether these low molecular weight compounds were indeed part of the purine degradation pathway. Commercially available purine standards were initially processed individually and were then subsequently compared to a calibration mixture to identify each compound based on its elution time. Chromatograms depicting the elution sequence of these purinogenic compounds at 254 nm and 280 nm are shown in FIG. 4. Comparison of the two profiles revealed that hypoxanthine, xanthine, adenosine and inosine elicited strong absorbance at 254 nm. In contrast, uric acid strongly absorbed at 280 nm rather than 254 nm.

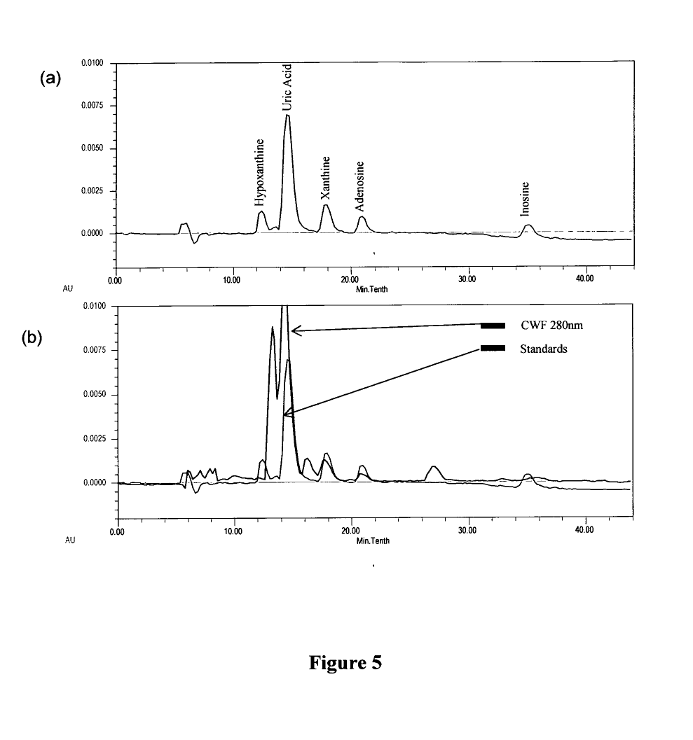

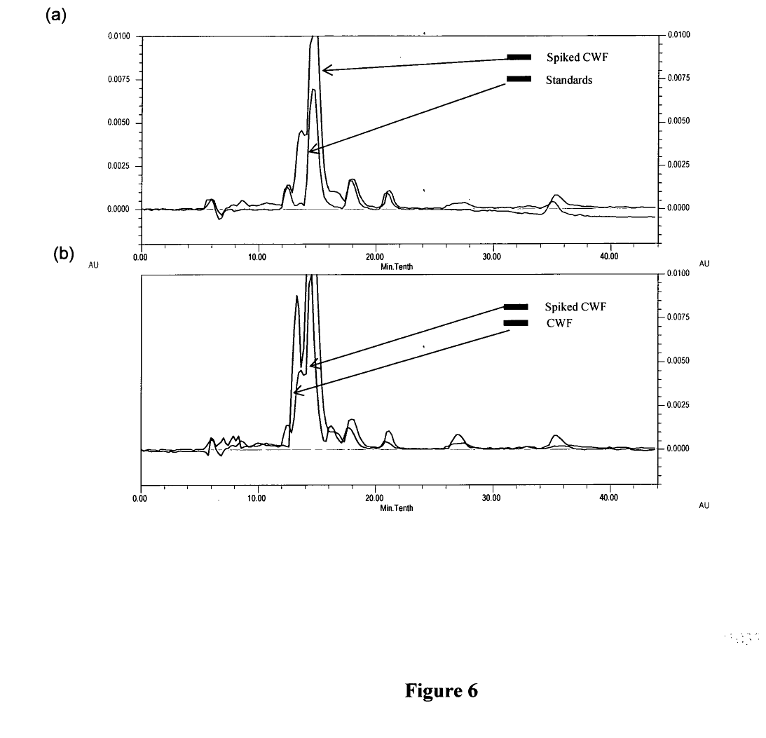

In order to confirm the presence of purine degradation products in wound fluid, small concentrations of each purine standard were incorporated into CWF. The amount of standard added to CWF was determined based on the absorbance values of CWF at 280 nm. A chromatogram depicting the amount of purine standards added to CWF is shown in FIG. 5a. Comparison of these chromatograms, as shown in FIG. 6b, provides a rough estimation of the retention times of each compound during separation.

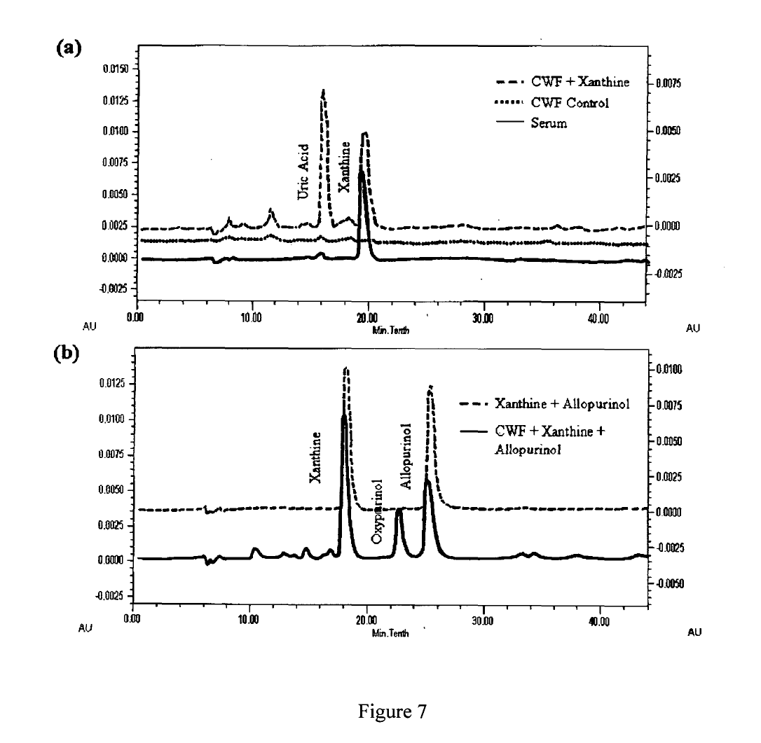

The predetermined amount of purine standards was added to CWF and analysed under similar conditions as previously described. Part (a) and (b) of FIG. 6 shows chromatograms that compare the spiked wound fluid with the add standards and CWF. Comparison of the added purine standards and spiked CWF chromatogram identifies the peak for each compound based on retention time (FIG. 7a). Taking this into consideration, the overlayed traces of CWF and spiked CWF suggest the presence of measurable amounts of purine metabolites xanthine and adenosine in CWF and with the predominant species being uric acid (FIG. 6b).

### Discussion

This study surprisingly provided evidence that suggests the presence of purine metabolites in chronic wound fluid collected from venous leg ulcer patients. These findings imply that two important changes occur in the chronic wound environment: firstly, the production of these purines indicates that the tissue is in fact ischemic in nature consequently resulting in the break down of ATP. Secondly, the presence of uric acid in wound fluid indicates that XO is active, oxidising hypoxanthine as well as xanthine.

In conclusion, we have provided evidence that suggests purine metabolites are elevated in CWF. Detection of these compounds in chronic wounds perhaps indicates that these tissues are hypoxic in nature which is likely due to poor tissue reperfusion. Thus, these purine compounds may be able to be used to predict the extent of tissue damage during inflammation and ischemia. In addition, the presence of uric acid in CWF suggests that XO is active in these tissues.

### Example 2

### Detection of Xanthine Oxidase in CWF

The activity of xanthine oxidase in a pooled CWF sample was determined by assessment of uric acid production at A₂₈₀. Additionally, samples were treated with allopurinol, a specific XO inhibitor, and the production of the metabolite oxypurinol determined at A₂₅₄. Firstly, CWF was concentrated by centrifugation using nanosep 3K omega filters (PALL) at 10,000 g for 10 min at 4° C. The retentate was resuspended with 10 mM Tris-HCl buffer, pH 8.0 containing either 10 μmol/L of xanthine or allopurinol, then incubated at 37° C. for 2.5 hr in the ultrafiltration unit. After incubation, the reaction mixture was de-proteinised as per the manufacturer's instructions and the resulting filtrates analysed using reverse phase HPLC as previously outlined. XO activity was expressed as amount of xanthine oxidised to uric acid per min per mg of total protein.

### Determination of Xanthine Oxidase Activity in Clinically Worse Ulcers

The activity of the oxidase form of XOR in wound fluid samples from clinically worse ulcers (n=10) was determined by measuring the oxidation of xanthine to uric acid spectrophotometrically at 295 nm. Briefly, CWF was concentrated by centrifugation using nanosep.3K omega filters (PALL) at 10,000 g for 10 min at 4° C. The retentate was resuspended with 10 mM Tris-HCl buffer, pH 8.0 containing 100 μmol/L of xanthine, then incubated at 37° C. overnight in the ultrafiltration unit. After incubation, the reaction mixture was de-proteinised as per the manufacturer's instructions and the resulting filtrates analysed using the NanoDrop ND-1000 (NanoDrop, Wilmington, Del., USA). XO activity was expressed as amount of xanthine oxidised to uric acid per mg of total protein.

### Results

### Evidence of XO in CWF

Enzyme activity was determined by supplementing wound fluid with the purine substrate xanthine and measuring the production of uric acid determined by HPLC at A₂₈₀. The resulting chromatogram as shown in FIG. 7 revealed an additional peak which was identified as uric acid as it exhibited a similar elution time as the commercially available uric acid standard. This oxidation of xanthine to uric acid was calculated to be approximately 58 nM of xanthine/min/mg of protein. In further support of this finding, XO enzyme inhibition was assayed using a potent XO inhibitor, allopurinol. Allopurinol is rapidly metabolised by XO to generate the active metabolite, oxypurinol, which itself is an inhibitor of XO. The results obtained did not reveal a peak corresponding to uric acid, however, a peak consistent with the retention time of oxypurinol standard was detected at A₂₅₄. Western blotting confirmed the presence of the enzyme in wound fluid (data not shown).

### Evidence of Xanthine Oxidase Activity in Clinically Worse Ulcers

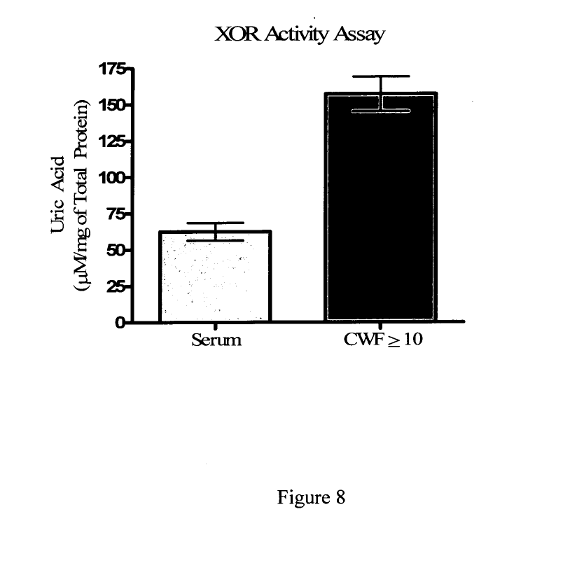

Xanthine oxidase activity was determined by supplementing wound fluid with the purine substrate xanthine and measuring the production of uric acid spectrophotometrically at 295 nm. FIG. 8 demonstrates elevated xanthine oxidase activity in 10 wound fluid samples obtained from clinically worse ulcers (PUSH 10-16) compared to human serum. The xanthine oxidase activity levels in the clinically worse ulcers were twice as much as those observed in human serum.

### Discussion

In this study, the presence of the oxidase form of XOR in chronic wound fluid through enzyme activity assays has been detected. These findings, together with the presence of the terminal purine catabolite, UA, imply that XOR is active within the wound margins, oxidising hypoxanthine, as well as xanthine. We postulate that the presence of elevated concentrations of XOR at the wound site is the source of uric acid in chronic wounds and leads to delayed healing.

### Example 3

### Simultaneous Detection of Purine Catabolites in Chronic Wound Fluid

Since chronic venous ulcers are normally hypoxic, the present inventors postulated that monitoring changes of these heterocyclic catabolites in a longitudinal study using CWF collected from patients may provide valuable information regarding the healing patterns of chronic venous ulcers. However, limited work has been done on the separation and quantification of these small heterocyclic catabolites in wound fluid. RP-HPLC has been the only technique proposed to date to simultaneously measure uric acid and its oxidation product allantion in wound fluid. However, there are uncertainties regarding the method employed to quantitate these metabolites with authors failing to outline specific details regarding validation and purine quantitation. More importantly, this technique lacks the sensitivity and specificity required to facilitate the detection of these low molecular weight purine catabolites.

For a number of years now, small molecule metabolites have been analysed with enhanced specificity and sensitivity using an ion monitoring technique, multiple reaction monitoring (MRM). This is a particularly robust technique mainly used to detect and quantitate a range of small molecule metabolites spanning from naturally occurring biological compounds to therapeutics, toxins, illicit drugs and their metabolites. Since its conception, the MRM technique has gone through improvement and has recently emerged as the method of choice for the analysis of proteins and peptides. This process identifies compounds of interest based on their specific fragmentation pattern at an assigned collision energy. The particular mass and structure of the targeted compound are used to predict the MRM transition, which is the precursor (m/z) and the product fragment (m/z).

Thus, the objective of the study was to firstly establish a specific and sensitive assay for monitoring low concentrations of the purine catabolites, adenosine, inosine, hypoxanthine, xanthine and uric acid, in chronic wound fluid. We therefore developed a procedure combining RP-HPLC with tandem mass spectrometry and MRM for the screening of these catabolites in CWF. Sequential CWF samples from both healing and non-healing patients were analysed using this technique to simultaneously separate and quantify purine catabolites. In addition to providing a new, reliable and efficient method of analysis, we provide evidence that indicates that these heterocyclic metabolites in CWF could potentially be used as diagnostic markers or therapeutic targets in the healing of chronic venous leg ulcers.

### Materials and Methods

### Materials

All general reagents and chemicals were analytical grade and sourced from a variety of companies. Purine metabolites including adenosine, inosine, hypoxanthine, xanthine and uric acid, as well as ammonium acetate were purchased from Sigma-Aldrich (St Louis, Mo., USA). Nanosep 3K omega centrifugal filtration devices were obtained from PALL (Pensacola, Fla., USA). All deionised water used in the following experiments was filtered by the Millipore system.

### Standard Solutions

Standard solutions of adenosine, inosine, hypoxanthine, uric acid and xanthine were prepared as described under the methods section in Chapter 5. Briefly, appropriate amounts of each purine were dissolved in 20 mmol/L NaOH to prepare stock standard solutions at 6 mmol/L. These standard solutions were stored at −20° C., and were thawed on ice prior to analysis. Equal volumes of all five standards were added and diluted with deionised distilled water to a working standard solution of 100 μM. This solution was further diluted to generate a calibration curve.

### Chronic Wound Fluid Samples

Wound fluid samples and ulcer assessment data were collected from patients with a venous ulcer. Wounds that were infected or samples that were contaminated with blood were excluded from this study. Clinical ulcer assessment data, included the PUSH score, ulcer size, duration and the amount of external compression applied at each time point.

CWF samples were thawed on ice before being de-proteinised using nanosep 3K molecular weight cut-off centrifugal filters (PALL) according to manufacturer's instructions. The resulting filtrates were then analysed using the 4000 QTRAP LC/MS/MS system (Applied Biosystems).

### HPLC/MRM Detection of Purine Catabolites

Standards and samples were analysed in triplicate using the fully automated UltiMate 3000 nano, capillary and micro LC system (Dionex, USA) which includes an autosampler. A Polaris C18 analytical column 5 μm, 250×4.6 mm i.d. (Varian) was used to fractionate purine catabolites in standard and sample solutions. The mobile phases used for these analyses were: buffer A, consisting of 10 mM ammonium acetate pH 4.7; and buffer B, containing a 1:1 mixture of buffer A and methanol. The purine catabolites were eluted from the column with a linear gradient from 100% buffer A to 100% buffer B over 5 min. After 2 min of isocratic elution with 100% buffer B, the mobile phase was switched back to 100% buffer A to regenerate the column. The length of the run was approximately 15 min with the flow rate maintained at 0.5 mL/min. A total volume of 20 μL of each sample was injected per run and introduced into the mass spectrometer at a rate of 10 μL/min.

Detection of purine catabolites was performed on the 4000 QTRAP LC/MS/MS system (Applied Biosystems), a triple quadrupole/linear ion trap mass spectrometer with a turbo ionspray interface which is ideal for metabolite identification (Hoke et al., 2001). The collision gas was nitrogen, the ionspray voltage was 4.5 kV, and the turbo spray temperature was maintained at 45° C. Purine catabolites were subsequently detected in the negative mode using tandem mass spectrometry with MRM. Transition ion pairs and declustering potentials were optimised for each analyte based on reference compounds. The transitions, declustering potential and collision energies for each compound are listed in Table 1.

### Results

### Separation of Purine Standards

For each purine compound, precursor and product ions were determined by directly injecting each purine standard into the QTRAP mass spectrometer. Identifying these precursor/product ion pairs allowed for enhanced selectivity for each purine compound in the MRM mode. The specific transitions obtained for each purine compound are listed in Table 1. Purine catabolites were then resolved by a linear gradient RP-HPLC and monitored by tandem mass spectrometry with negative electrospray ionisation (ESI-MS/MS). The HPLC-MRM run allowed for suitable separation of uric acid, hypoxanthine, xanthine, inosine and adenosine with approximate retention times of 10.4, 10.6, 10.9, 11.1 and 12.2 min, respectively (data not shown). Thus, this method was selectively capable of detecting each purine catabolite based on HPLC fractionation coupled with the analysis of separate MRM analysis.

### Calibration Curves

The calibration curves were determined over the range 0.25-10 μM by diluting the working standard solution in buffer A. These standards were subsequently analysed in triplicate using the MRM method described herein. Linear regressions were performed on the five purine catabolites with quantitation based on the peak area counts of each compound. The calibration curves show a linear response with correlation coefficients (r) between (0.9978-0.9996) for all catabolites (data not shown). The detection limit was classified as a signal to noise ratio of 3.

### Recovery Analysis

The assessment of the analytical recovery was conducted by preparing two biological samples. The first, a reference sample, comprised of pooled wound fluid and the second was the same sample spiked with standard solutions at two concentrations, 10 μM and 2.5 μM, prior to filtration. Recovery was subsequently determined by subtracting the value of the reference sample from that in the spiked wound fluid sample. The extraction yields for the different purine compounds were calculated to be in the range of 60-125% as shown in Table 2. The recoveries for adenosine were slightly lower than expected, with 60% and 67%, respectively, as indicated in Table 2.

### Uric Acid an Important Indicator of Wound Chronicity

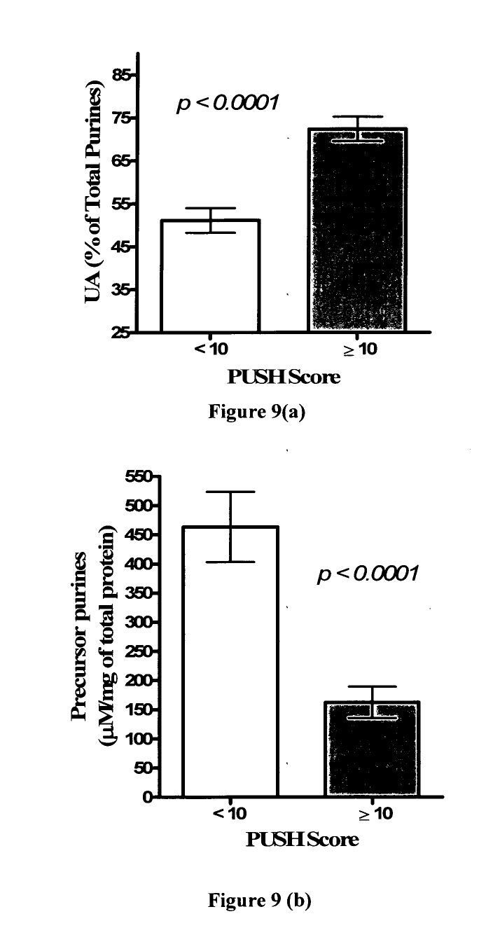

In order to verify these findings the median PUSH score for the 55 wound fluid samples analysed was calculated to be 10 (Range 3-16). Chronic wound fluid samples were then split into two groups: PUSH scores 10-14 (n=26) representing the clinically worse ulcers and 4-9 (n=29) the less severe ulcers. Firstly, the amount of UA as a percentage of total purines was assessed against the two groupings of PUSH scores as indicated FIG. 9(a). The results obtained imply that the amount of UA detected was significantly increased in the clinically worse ulcers (10-14) compared to the lower PUSH score group (4-9).

### Reduced Levels of Purine Precursors in Clinically Worse Ulcers

The amount of purine precursors including adenosine, inosine, xanthine and hypoxanthine were also assessed against the PUSH score as shown in FIG. 9(b). The results obtained suggest that the amount of precursor purines were significantly higher in the lower PUSH score group (4-9) compared to the clinically worse ulcers (10-14). This implies that there are significantly higher levels of purines that serve as substrates for the enzyme XO in CWF obtained from patients assigned a lower PUSH score compared to a higher PUSH score.

### Discussion

The previous analysis of CWF presented above led to the detection of a family of heterocyclic compounds in CWF that are part of the purine catabolic pathway. The present inventors are of the view that these purine catabolites may potentially function as indicators to guide venous ulcer management and therefore it would be worthwhile examining the expression of purines in sequential wound fluid samples collected from patients with both healing and non-healing venous ulcers. Our initial examination of CWF was based on a simplistic RP-HPLC technique for the separation of purinogenic compounds. Fractionation of these metabolites in CWF was non-specific and prone to interferences by other UV absorbing molecules. In addition, no work has been reported previously on the separation of the uric acid precursors, adenosine, inosine, hypoxanthine and xanthine, in CWF.

In the study reported in Example 3, the inventors have developed and validated a reliable, simple and specific analytical assay for separation and simultaneous monitoring of low concentrations of purine catabolites in CWF. The combination of RP-HPLC with MS/MS enabled specific analysis of these potential indicators of healing by MRM. This procedure was successfully validated and subsequently applied to determine the levels of purine catabolites in sequential wound fluid samples. The wound fluid collection technique employed for our study was the most suitable for the isolation of these purine catabolites from the wound environment. In addition, the application of saline to the wound area prior to collection also ensured that the entire wound site was assessed. However, in order to account for the dilution of these catabolites during sample collection, purine concentration was normalized to wound fluid protein content. The only inconsistency detected was that the extraction yields obtained for adenosine were surprisingly lower than expected (Table 2). This may reflect the greater instability of adenosine once added to CWF relative to other purines. One possible explanation for this anomaly is that adenosine is converted to catabolites further down the purine degradation pathway. Interestingly, the inosine recoveries values obtained were >100% which may support this theory.

As noted earlier, elevated levels of UA in the chronic wound environment are likely to play an important role in healing. The excessive accumulation of UA in clinically worse ulcers (FIG. 9(a)) suggests that XOR is active in the wound site releasing toxic free radicals in the process. This is further supported by our finding that there are elevated levels of purine precursors in wound fluid from patients with less severe ulcers (FIG. 9(b)). Moreover, it can be postulated that xanthine oxidase activity could also be a factor in wound severity due in part to the finding that xanthine oxidase activity is inhibited by allopurinol in CWF (as hereinbefore described). Therefore, wound severity may well be related to XOR catalysis of purine precursors to UA.

Physiologically the solubility of UA in human serum is 404 μmol/L at pH 7.4 and 37° C. (Loeb, 1972). Sustained production of UA in underperfused damaged tissues may result in the precipitation of UA as is commonly observed in the case of gout (McCarty and Hollander, 1961). Therefore, a slight drop in pH or temperature could lead to the deposition of sodium urate crystals around the wound environment. A characteristic feature of chronic venous leg ulcers is the paucity of blood supply to the wound bed, which results in a switch to anaerobic metabolism and the build up of lactic acid. Impaired elimination of lactic acid from the tissue results in a decrease in pH and a consequential decrease in the solubility of sodium urate. Interestingly, suspensions of monosodium urate crystals have been previously shown to be capable of producing an inflammatory response in both gouty and non-gouty patients. Furthermore, these induced attacks were dramatically subdued on administration of therapeutic doses of an uric acid inhibitor (Seegmiller et al., 1962). Accumulation of these crystals in an already inflamed area may therefore provide added stimulus resulting in an even greater inflammatory response. Thus, uric acid may serve as an important diagnostic indicator for management of these lesions and critically may also be as a therapeutic target for the treatment of venous ulcers.

Currently wound prognosis is rather subjective, relying largely on clinical signs and the clinician's experience and knowledge in the field. Misdiagnosis and improper treatment can compromise healing, resulting in the formation of a static, non-healing ulcer. Currently, differential diagnosis is based on visual wound assessment along with other measures such as ABPI which essentially determine the origin or type of ulcer rather than predict the healing trajectory of the wound. While research has improved our understanding of chronic wound healing at a cellular and molecular level, to date no reliable molecular or biochemical indicators of healing or non-healing have been reported.

The analytical method established in this study allows for rapid and selective screening of purine metabolites in CWF. However, this technique is expensive and impractical for monitoring or diagnosing chronic wounds in a clinical setting. A diagnostic test that is quick, easy to use and can be implemented at the bedside would be more beneficial. Nevertheless, the results provides a promising method for diagnosis by targeting one or more members of the purine catabolic pathway that lead to UA production. Development and implementation of point of care testing based on these findings could be used to monitor the onset, prognosis and progress of chronic venous leg ulcers. More importantly, the research presented in this study also provides new options for treating these lesions through the inhibition of xanthine oxidase and/or UA production in wounds. Taken together, the data indicates that hypoxia induced purine catabolism is an underlying process implicated in the formation of chronic wounds.

### CONCLUSION

In summary, the data presented in this study describes the establishment of a specific and sensitive assay for monitoring low concentrations of the purine catabolites: adenosine, inosine, hypoxanthine, xanthine and uric acid, in CWF. This technique was successfully applied to simultaneously separate and quantify purine catabolites in sequential CWF samples collected from both healing and non-healing wounds. The results obtained strongly imply that changes in purine catabolite profiles in wound fluid correlates to wound chronicity. In particular, the amount of UA was elevated in wound fluid collected from clinically worse wounds in comparison to CWF collected from less severe wounds. The accumulation of UA indicates that XOR is catalysing the oxidation of purine substrates thereby generating increased amounts of free radicals. This was further supported by the detection of decreased levels of precursor purines in the CWF collected from severe wounds. Based on the current findings these heterocyclic compounds could potentially be used as diagnostic markers for monitoring wound prognosis and response to treatment. The development of non-invasive point of care testing that targets one or more members of the purine catabolic pathway that lead to uric acid production would assist with wound diagnosis. More importantly, the development of a specific diagnostic tool would transform current wound management practices by guiding clinical decisions enabling practitioner's to diagnose wound status more accurately and in turn, provide effective customised treatments that would reduce healthcare costs and improve wound healing outcomes.

### Example 4

### Inhibition of XOR Via Topical Application of Allopurinol will Promote Healing of Chronic Venous Leg Ulcers will be Examined as Follows

1. Conducting a randomised study using standardised techniques to measure the physical progress of wound healing in patients receiving topical allopurinol or a placebo control;

2. Characterising longitudinal biochemical changes in wound fluid collected across the 2 treatment groups during wound healing progression. Specific biochemical indicators used to measure the efficacy of the treatments will include:
(A) Purine profiles including the relative concentrations of adenosine, inosine, hypoxanthine, xanthine and UA;
(B) XOR activity and protein levels;
(C) Redox potential as determined by potentiometry; and
(D) Profiles of key small molecular weight redox couples including glutathione, homocysteine and cysteine.

3. Determining relationships between progress in wound healing and the biochemical changes in wound fluids across the 2 treatment groups.

### 1: Conduct a Clinical Trial to Evaluate Allopurinol as a Treatment for Chronic Venous Leg Ulcers

Study Design: A randomised controlled pilot double blinded clinical trial will be conducted to examine the physical and biochemical effects of topical allopurinol. A random allocation sequence will be generated by the project coordinator using a computer randomisation program prior to commencement of recruitment.

Patients with confirmed venous leg ulceration will be recruited and randomly assigned to two treatment groups:
Treatment Group A: 30 mg allopurinol formulated in 2.0% (w/v) carboxymethyl-cellulose (CMC) in Phosphate buffered saline (PBS)
Treatment Group B: Placebo: 2.0% (w/v) CMC in PBS

Sample Size: A total of 70 participants (35 per group) would be required based on:

A sample of 29 completing subjects per group will be required to detect a 30% difference between groups in mean percentage reduction in ulcer area (e.g. 80% mean percentage reduction in group A vs. 50% in group B) after 12 weeks from intervention and allowing for 20% attrition. Sample size will be determined by power analysis and based on expected differences in healing (from previous studies) between groups (significance level of 0.05, power 90%).

Preparation of Test Treatments:

The XOR inhibitor allopurinol is a registered medication (>30 years) that is commonly prescribed for the long-term treatment of gout and hyperuricemia. As a gout treatment, allopurinol is delivered orally at 200 to 300 mg/day for patients with mild gout and 400 to 600 mg/day for those with moderately severe tophaceous gout. Allopurinol is well tolerated and has low toxicity. In mice, the 50% lethal dose (LD50) is 700 mg/kg when given orally and 160 mg/kg when given intraperitoneally. The formulations proposed in this study, 30 mg/mL/week will be delivered topically, a tenth of the commonly used gout treatment dosage. Moreover, this dose will be applied topically weekly, rather than orally daily. Of note, a number of studies have reported positive results with the use of topical allopurinol in the treatment of corneal alkali burns and in patients with radiation-induced mucositis and dermatitis.

Bulk solutions of the two treatments listed above will be prepared under sterile conditions and 1 mL aliquots will be dispensed in treatment tubes. These tubes will be terminally sterilised using 50 kGy gamma irradiation and stored at −80° C. until administration. Accelerated stability trials will be conducted to demonstrate stability across a range of temperatures and storage periods using established HPLC methods for monitoring allopurinol degradation. Tubes from each treatment group will be coded prior to delivery to the clinic and each tube will be labelled according to the patient number. Thus, the nature of each treatment will be unknown to those applying the treatments and measuring the treatment outcomes, as well as to the patient.

Treatment & Sampling:

Following recruitment, baseline ulcer and patient data will be obtained and all patients will be treated once a week across a 12 week period in combination with standardised compression dressings giving 40 mmHg at the ankle. CWF will be collected according to our standard protocol prior to the topical application of the treatments. In brief, wound fluid collection involves the application of an occlusive dressing over the wound site and CWF is collected after 1 h. Despite the likely importance of redox status and pH in the chronic wound it is rarely examined in clinical settings. Therefore, in this project pH, as well as redox potential, will be measured using potentiometry. Due to our interests in measuring free thiol concentrations and the redox status, the CWF collection protocol will be modified to incorporate the use of BioPool Stabilyte™. Prior studies have demonstrated that Stabilyte™ is capable of stabilising the oxidation of free thiols of whole blood for up to 8 h at 18-23° C. and extended periods at −70° C. Consequently, the pH and redox potential of the exudate will be measured immediately after aspiration using an oxidation-reduction electrode (ORP-146C Micro, Lazerlab) and before the addition of Stabilyte™. Samples will be centrifuged at 14,000 g for 10 minutes to remove particulate debris, and will be filter-sterilised and stored at −80° C. until further analysis.

Patient Assessments:

Ulcer healing assessment data will be collected every week by the on-site research nurse from enrolment in the study until 24 weeks from recruitment (or until the ulcer has healed if this is earlier). Physical progress in wound healing will be measured with the following “best practice” methods:
Ulcer area will be calculated by using wound tracings, digital photography and Visitrak™ (Smith and Nephew) to determine area reduction, percent reduction and total healing rates;
The PUSH tool for ulcer healing will also be used to provide a more sensitive measure of healing than examining area alone. The PUSH score incorporates wound area, exudate and the type of tissue (i.e. epithelial, granulating, slough or necrotic);
Clinical data related to healing progress, such as presence of oedema, eczyma, inflammation, signs of infection and information on dressing and compression type, will be collected.

### 2: Characterise Biochemical Changes in Wound Fluid Collected Across the Treatment Groups During Wound Healing Progression.

Purine Profiling:

We have recently developed and validated a novel, sensitive and highly specific analytical method for quantifying purines in CWF. This procedure involves combining HPLC with tandem mass spectrometry and multiple reaction monitoring (MRM). MRM is used to detect the specific fragmentation pattern of a substance at an assigned collision energy. The mass and structure of the compound are used to predict the precursor (m/z) and the product fragment (m/z) (MRM transition) for that particular compound. This application is selective, using specific MRM transitions and HPLC separations for each analyte, making it ideal for the detection and quantification of metabolites in CWF. This approach will be used to monitor longitudinal changes in purine levels across each patient and will provide valuable information which will be analysed for correlation with static, fluctuated or healing patterns of chronic venous ulcers. The purines that will be measured in this study include adenosine, inosine, hypoxanthine, xanthine and UA. In brief, samples will be analysed using the fully automated UltiMate 3000 nano, capillary and micro LC system (Dionex, USA) which includes an autosampler and a Polaris C18 analytical column 5 μm, 250×4.6 mm i.d. (Varian). Purinogenic compounds will be detected with the 4000 QTRAP LC/MS/MS system with turbo ionspray interface (Applied Biosystems), a triple quadrupole/linear ion trap mass spectrometer that is ideal for metabolite identification. Purine metabolites will be detected in the negative mode using tandem mass spectrometry with MRM using established transitions, declustering potential and collision energies for each compound. Longitudinal analysis of the purine profiles across the treatment groups will confirm the pharmacological action of allopurinol through elevated concentrations of adenosine, inosine, xanthine and hypoxanthine with corresponding reductions in UA concentrations.

Low Molecular Weight Thiols:

Disruption of redox homeostasis in a particular tissue or biological fluid is considered to be a critical factor in the pathogenesis of many disease states. Redox in-balance in the extracellular environment can disrupt inter- and intra-molecular disulfide bonds important for stabilising protein structure and the regulation of protein function. The ratio of glutathione (GSH) to its corresponding oxidised form (GSSG) is commonly used as an effective measure of oxidative stress. Similarly, homocysteine (HCysSH), cysteine (CysSH) and their corresponding oxidised forms, homocystine and cystine, represent important physiological redox couples. Both reduced and oxidised forms of GSH, HCysSH and CysSH will be measured as general indicators of oxidative stress and as potential prognostic indicators of wound healing and will compare this to the PUSH tool measure of ulcer healing. The experimental approach will be similar to that employed for measuring purine concentrations. In brief, wound fluid samples will be derivatised with Ellman's reagent and total protein will be precipitated with sulfosalicylic acid using standard approaches. Following the removal of protein by centrifugation, supernatants will be recovered and stored at −80° C. prior to LC/MS/MS analysis on a 4000 QTRAP. Similar to that described by Guan et al. 2003, a MRM approach will be used to simultaneously monitor ions with m/z of 269, 613, 319, 333 and 505 which correspond to the protonated molecular ions of homocystine, GSSG and Ellman's reagent derivatised CysSH, HCysSH and GSH.

Xanthine Oxidoreductase Inhibition:

To further characterise the efficacy of topical allopurinol administration, XOR activity and protein levels will be measured for all wound fluid samples using our established assays. In brief, wound fluid will be concentrated using Nanosep 3K omega ultrafiltration filters and the retentate will be incubated with 10 μM xanthine at 37° C. for 1 h. Following incubation, the reaction mixture will be deproteinised and the conversion of xanthine to UA will be measured using reverse-phase HPLC. XOR activities will be expressed and compared as the amount of xanthine oxidised to UA per hour per mg of total protein. Total XOR levels will be monitored using a standard Western blot protocol based on a commercially available anti-XOR polyclonal antibody (Santa Cruz Biotechnology).

### 3: Determine Relationships Between Progress in Wound Healing and the Biochemical Changes in Wound Fluids Across Treatment Groups.

Statistical Analysis:

SPSS Statistical Software (Version 17) will be used for all statistical analyses. Data analysis will follow “intention to treat” guidelines and will follow these steps:

1. Baseline variables from both groups will be analysed to check for similarity of the groups. Any clinically meaningful differences detected will be controlled for during the subsequent analysis.

2. Descriptive statistics will be calculated for each primary outcome measure.

3. Data on key prognostic factors (e.g. ulcer size >10 cm², duration >24 wks, level of mobility, age) will be collected and these variables controlled for during analysis.

4. Comparisons between groups will be undertaken. ANOVA and Kruskal

Wallis tests will be used for analysis of ulcer healing measures.

5. Repeated measures regression modelling using generalised estimating equations will investigate differences over multiple data collection points and determine the impact of interventions over time.

6. Kaplan-Meier survival curves will be used to compare the two groups' time to complete ulcer healing. A log rank statistic will be used to test the hypothesis that the cumulative readmission-free rate curves are identical.

7. A Cox proportional hazards regression model will be used to mutually adjust for any potential confounding variables and determine the effect of topical allopurinol on healing.

8. Data generated from the purine and thiol profiling will be analysed using one-way ANOVA to establish a correlation between these biochemical indicators and wound healing.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

### Tables

TABLE 1
MRM transitions for the detection of purine metabolites in CWF

| | | Precursor | | | |
|---|---|---|---|---|---|
| | | Ion | Product | Declustering | Collision |
| Compound | Mᵣ | m/z | Ion m/z | Potential | Energy |
| Adenosine | 267.2 | 266 | 134 | 30 | 23 |
| Hypoxanthine | 136.1 | 135 | 92 | 25 | 23 |
| Inosine | 268.2 | 267 | 135 | 30 | 23 |
| Xanthine | 152.1 | 151 | 108 | 25 | 23 |
| Uric Acid | 168.1 | 167 | 124 | 23 | 23 |

TABLE 2
Recoveries of Purine Catabolites in CWF

| | 10 μM Purine | | | |
|---|---|---|---|---|
| | Standards | | 2.5 μM Purine Standards | |
| | Mean | | Mean | |
| | Recovery | | Recovery | |
| Compound | (μM) | Recovery % | (μM) | Recovery % |
| Adenosine | 6.7 | 67 | 1.5 | 60 |
| Hypoxanthine | 10.5 | 105 | 2.7 | 108 |
| Inosine | 12.5 | 125 | 3.1 | 124 |
| Xanthine | 8.6 | 86 | 2.2 | 88 |
| Uric Acid | 8.7 | 87 | 2.1 | 84 |

1. A method of treating a wound, ulcer and/or burn responsive to inhibition of xanthine oxidoreductase and uric acid in an animal in need of such treatment, said method including the step of topically administering to said wound, ulcer and/or burn a therapeutic agent effective for treatment of the wound, ulcer and/or t burn, wherein said therapeutic agent inhibits xanthine oxidoreductase to thereby reduce uric acid in the wound, ulcer and/or burn, and thereby treat the wound, ulcer and/or burn in said animal.

2. The method of claim 1, wherein xanthine oxidoreductase is the xanthine oxidase form.

3. The method according to claim 1, wherein the therapeutic agent is a selective inhibitor.

4. The method according to claim 1 wherein the therapeutic agent is selected from the group consisting of a small organic molecule, an isolated protein, an isolated peptide and an isolated nucleic acid.

5. (canceled)

6. The method according to claim 5, wherein the therapeutic agent is a purine analogue.

7. The method according to claim 6, wherein the purine analogue is a xanthine analogue, a hypoxanthine analogue, or a flavonoid.

8. The method according to claim 7, wherein the xanthine analogue is selected from the group consisting of allopurinol, adenosine, oxypurinol and thiopurinol.

9. (canceled)

10. The method according to claim 6, wherein the hypoxanthine analogue is alloxanthine.

11. (canceled)

12. The method according to claim 7, wherein the flavonoid is selected from the group consisting of febuxostat, myricetin, kaempferol and quercetin.

13. The method according to claim 1, wherein the therapeutic agent has an IC₅₀ of less than 1 mM.

14. The method according to claim 1, wherein the wound is a diabetic wound.

15. The method according to claim 1, wherein the ulcer is selected from a pressure ulcer and a venous ulcer.

16. The method according to claim 15, wherein the venous ulcer is a venous leg ulcer.

17. (canceled)

18. (canceled)

19. The method according to claim 1, further including the step of determining whether the animal has an elevated level of xanthine oxidoreductase and/or uric acid in the wound, the ulcer and/or the burn.

20. The method according to claim 1, wherein the method further includes the step of promoting clearance of uric acid.

21. The method according to claim 20, wherein the step of promoting clearance of uric acid includes application of a physical stimulus and/or a chemical stimulus.

22. The method according to claim 21, wherein the physical stimulus is compression.

23. The method according to claim 22, wherein the physical stimulus is applied by a compression bandage.

24. The method according to claim 21, wherein the chemical stimulus is an enzyme which degrades uric acid.

25. The method according to claim 24, wherein the enzyme is uricase.

26. A method of determining the severity of a wound, a burn and/or an ulcer in an animal, said method including the step of detecting one or more of the following:
(i) a level of uric acid, wherein an increased level correlates with increased wound severity;
(ii) a level of one or more uric acid precursors, wherein a decreased level correlates with increased wound severity; and
(iii) a presence or absence of, or level of, xanthine oxidoreductase, wherein an increased level correlates with increased wound severity
in a cell, a tissue and/or a fluid of the wound, the burn and/or the ulcer to thereby determine the severity of the wound, the burn and/or the ulcer.

27. The method of claim 26, wherein xanthine oxidoreductase is the xanthine oxidase form.

28. The method of claim 26, which detects (i) and (ii).

29. The method according to claim 26, which correlates a ratio of (i) relative to (ii) with increased wound severity.

30. The method according to claim 29, wherein the ratio is an elevated ratio.

31. (canceled)

32. (canceled)

33. A kit for determining the severity of a wound, a burn and/or an ulcer, said kit comprising one or more reagents for detecting one or more of the following:
(i) a level of uric acid, wherein an increased level correlates with increased wound severity;
(ii) a level of one or more uric acid precursors, wherein a decreased level correlates with increased wound severity; and
(iii) a presence or absence of, or level of, xanthine oxidoreductase, wherein an increased level correlates with increased wound severity,
in a cell, a tissue and/or a fluid of the wound, the burn and/or the ulcer to thereby determine the severity of the wound, the burn and/or the ulcer.

34-55. (canceled)
